(19) United States
(12) Patent Application Publication
(10) Pub. No.: US 2009/0166560 A1
(43) Pub. Date: Jul. 2, 2009

(54) Sensing of biological molecules using carbon nanotubes as optical labels

(75) Inventors: Hongjie Dai, Cupertino, CA (US); Sarunya Bangsaruntip, Mount Kisko, NY (US); Nozomi Nakayama, Mountain View, CA (US)

Correspondence Address: PETERS VERNY , L.L.P., 425 SHERMAN AVENUE, SUITE 230, PALO ALTO, CA, 94306, US

(73) Assignee: The Board of Trustees of the Leland Stanford Junior University

(21) Appl. No.: 11/977,550

(22) Filed: Oct. 24, 2007

Related U.S. Application Data
(60) Provisional application No. 60/854,552, filed on Oct. 26, 2006.

(51) Int. Cl. G21G 5/00; G01J 3/44; G01N 33/566

(52) U.S. Cl. 250/492.1; 356/301; 435/7.1

(57) ABSTRACT

Disclosed are methods and materials including carbon nanotubes which have a strong Raman and/or fluorescent signal and which have been modified with an amphiphilic molecule having available functional linking groups for linking to a biological compound. Exemplified are surface-functionalized SWNTs (single walled nanotubes) as highly sensitive bio-labels based on the detection of their spectroscopic Raman signature. By solubilizing the nanotubes with polyethylene glycol (PEG)-containing phospholipids, aqueous-stable as well as biocompatible SWNT labels are produced. Specificity in biological detection is then attained by immobilizing reporting molecules off this PEG layer. Highly selective detection of surface immobilized proteins is achieved with detection limit of ˜10 femtomolar, three orders of magnitude higher than the fluorescent technique. Signal stability upon Raman readout as well as compatibility of the SWNT-tagged proteins to the microarray protocols are also demonstrated, making these biocompatible SWNTs highly attractive as novel, alternative bio-labels for ultrasensitive detection of proteins. When excited with a near infrared laser, the nanoparticles give off a distinctive fluorescence signal.

### CROSS-REFERENCE TO RELATED APPLICATIONS

This application claims priority from U.S. Provisional Patent Application No. 60/854,552, filed on Oct. 26, 2006, which is hereby incorporated by reference in its entirety.

### STATEMENT OF GOVERNMENTAL SUPPORT

This invention was made with U.S. Government support under NIH Grant 1 U54 CA 119367-01. The U.S. Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the field of optical labels (Raman, fluorescent, etc.), which can be used to label biological materials.

2. Related Art

In recent years, biological applications of carbon nanotubes have been intensely investigated, and they have been successfully employed in various applications such as electronic biosensors,¹⁻³ molecular transporters for cargo delivery into cells,⁴⁻⁶ and substrates for neuronal growth and signaling.^{7,9} Single-walled carbon nanotubes (SWNTs) have a unique Raman spectrum; additionally, they can be solubilized in aqueous environments by amphiphiles. We have found that biological molecules can be attached to these aqueous-stabilized SWNTs, and the interactions to their complements can be followed by the Raman signature of the SWNTs—thus allowing the use of carbon nanoparticles such as SWNTs as novel labels. While SWNTs have been previously solubilized in aqueous environments¹⁰⁻¹² and biomolecules have been attached to SWNTs,¹²⁻¹⁴ the present materials and methods are believed to be the first report on exploiting SWNTs as labels based on their Raman signature.

SWNTs have a strong Raman signal due the resonance enhancement, and individual SWNTs can be easily detected.¹⁵ This allows high detection sensitivity of the SWNT-labeled biomolecules (at the femtomolar level) without further need for subsequent signal amplification steps. Therefore, this novel label outperforms traditional fluorescent, radioactive, and enzymatic tags (limited to picomolar levels). The detection of SWNT-labeled biomolecules is also highly selective because biological molecules do not show competing Raman backgrounds. Furthermore, the Raman signals of the SWNT-labels are stable overtime, in contrast to the aforementioned labels, permitting measurements to be performed with high accuracy and reproducibility as described below, other carbon nanoparticles have strong Raman signals and may be used according to the present methods.

SWNTs also have been shown to have near infrared (NIR) fluorescence properties under certain circumstances. Their NIR photoluminescence (PL) lies within the “biological window” (700-1300 nm), where absorption, scattering, and autofluorescence by tissues, blood, and water are minimized. See, Choi et al., Multimodal Biomedical Imaging with Asymmetric Single-Walled Carbon Nanotube/Iron Oxide Nanoparticle Complexes, Nano Letters, 2007 Vol. 7, No. 4 861-867.

### Specific Patents and Publications

US 20050147976 to Su, published Jul. 7, 2005, entitled “Methods for determining nucleotide sequence information,” discloses a nucleic acid sequencing method based on detection of Raman signatures of oligonucleotide probes. Known chemical Raman labels may be used, such as TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET(6-carboxy-2′,4,7,7′-tetrachlorofluorescein), HEX(6-carboxy-2′,4,4′,5′,7,7′-hexachlorofluorescein), Joe (6-carboxy-4′,5′-dichloro-2′,7′-dimethoxyfluorescein)5-carboxy-2′,4′,5′,7-′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g., Cy3, Cy3.5, Cy5), xanthines, succinylfluoresceins, N,N-diethyl-4-(5′-azobenzotriazolyl)-phenylamine and aminoacridine.

US 20050244325 to Nam, et al., published Nov. 3, 2005, entitled “Carbon nanotube, electron emission source including the same, electron emission device including the electron emission source, and method of manufacturing the electron emission device,” discloses carbon nanotube (CNT) with Raman spectrum having a G band and a D band, and includes a ratio of a G band peak integral IG and a D band peak integral ID which is 5 or greater.

U.S. Pat. No. 5,814,516 to Vo-Dinh, issued Sep. 29, 1998, entitled “Surface enhanced Raman gene probe and methods thereof,” discloses the use of a gene probe biosensor and methods thereof based on surface enhanced Raman scattering (SERS) label detection.

U.S. Pat. No. 5,445,972 to Tarcha, et al., issued Aug. 29, 1995, entitled “Raman label and its conjugate in a ligand-binding assay for a test sample analyte,” discloses Surface Enhanced Resonance Raman Scattering (SERRS) as applied to immunodiagnostics. It is disclosed that a specific binding member coupled to a Raman active label and bound near the surface of a glass can potentially exhibit an even more pronounced SERRS effect than in conventionally used surfaces.

Bachilo et al., “Structure-Assigned Optical Spectra of Single-Walled Carbon Nanotubes,” Science, 298:2361-2366 (December 2002) discloses the use of Raman data to determine SWNT structure.

### BRIEF SUMMARY OF THE INVENTION

The following brief summary is not intended to include all features and aspects of the present invention, nor does it imply that the invention must include all features and aspects discussed in this summary.

The present invention provides compositions useful for labeling biological molecules, which may be present in low concentration. A composition comprises a carbon nanoparticles, which has a distinct Raman signal of high intensity compared to background; a solubilizing molecule, such as an amphiphilic molecule having a hydrophobic portion attached to the carbon nanotube and a hydrophilic portion for solubilizing the composition and having a linking group, so that the nanoparticles can be prepared in an aqueous environment; and a biological labeling molecule attached to the linking group, which labeling molecule attaches specifically to an analyte of interest. In a preferred embodiment, the carbon nanoparticle is a carbon nanotube, and specifically, a single walled carbon nanotube. The Raman signal enhancement is preferably realized through a nanoparticle having an sp2 carbon bonding structure. The amphiphilic molecule attached to the nanoparticles for solubilization may comprise a phospholipid, where an aliphatic or lipid portion is adsorbed onto the nanoparticles, and the phosphate head group is bound to a hydrophilic polymer such as PEG.

The biological labeling molecule is a biological molecule that has a specific binding to a target. Examples are proteins (such as antibodies, binding to antigens, enzymes, binding to substrates, receptors binding to ligands) and polynucleotides where specific binding is mediated by base pairing rules.

In another aspect, the present invention involves a method for preparing a composition for labeling a biological material. One uses a nanoparticle as described above, comprising the steps of contacting the nanoparticle with a solubilizing molecule; and linking a biological labeling molecule to the functionalized hydrophilic portion. The method involves preparation of nanoparticles, such as HIPCO production of SWNTs. Other nanotube production techniques may be used, such as arc discharge or laser ablation methods. As part of the present process, the nanoparticles (SWNTs) are sonicated with the solubilizing molecule (e.g., phospholipid-PEG), then additional hydrophilic polymer may be added. This step reduces the size of larger particles. The solubilized nanoparticles is then coupled to a biological labeling molecule (e.g., antibody), after deprotection of any protective groups on a reactive and of the solubilizing molecule. In one embodiment, the biological labeling molecule is an antibody to IgG. This provides a universal sandwich assay, where detection of bound IgG is detected. For example, if the primary label is a mouse IgG to a specific analyte, the Raman-labeled antibody will be against any mouse IgG.

In another aspect, the present invention involves the use of a thiolated antibody, which is linked to a bifunctional coupling agent that has been bound to the solubilizing molecule.

In another aspect, the present invention provides a method of detecting an analyte using Raman scattering with an optical source exciting a sample and an optical detector detecting changes in light scattered by the sample, comprising the step of labeling a portion of the sample with a specific ligand linked to a carbon nanoparticle. One may detect a Raman shift around 1600 cm-1, which is characteristic of an SWNT.

Another aspect of the present invention is the use of different nanoparticles with different Raman signatures. For example, different carbon nanotubes with different diameters and chiralities will have different RBM modes. Different antibodies (or other specific labeling molecule) can be attached to different types of nanotubes (e.g., MWNT and SWNT, or metallic or semiconducting SWNT, or MWNTs of different diameters) in order to obtain a multiplex signal, by assaying two different analytes in the same process. In fact, numerous different analytes can be simultaneously assayed in this way, owing to the breadth of the potential Raman spectrum.

Another aspect of the present invention comprises a method of labeling a cell for detection by fluorescence, using solubilized carbon nanoparticles, e.g., as referred to above. That is, the carbon nanoparticle is comprised in a composition in which the carbon nanoparticle is complexed to an amphiphilic polymer, e.g., phospholipid-polyethylene glycol (PEG), and the hydrophilic portion of the polymer is attached to a biological labeling molecule, such as an antibody. The antibody serves to direct the complex to an antigen to be bound for labeling. Thus, the process may comprise the steps of: contacting the cell with a composition comprising (i) a carbon nanoparticle; (ii) a solubilizing molecule having a hydrophobic portion attached to the carbon nanoparticle and a hydrophilic portion for solubilizing the composition and having a linking group; and (iii) a biological labeling molecule specific for the cell attached to the linking group. This composition is contacted with a particular cell to be labeled, in that the cell contains a recognition site specific for the labeling molecule. Thus, the complex binds to the cell to be labeled. The complex is exposed to near infrared light, e.g., by irradiating with an NIR laser, e.g., at about 785 nm wavelength. One then detects near infrared photoluminescence from the carbon nanoparticle, which will only emanate from the labeled cell, since biological tissues are generally transparent to NIR radiation. The carbon nanoparticle may be a carbon nanotube (SWNT or MWNT) and the photoluminescence that is emitted may be in the 900-1600 nm region. Since the fluorescence detecting is done at an irradiating wavelength that is also suitable for Raman detection, both detections may be carried out on the same labeled cell.

### BRIEF DESCRIPTION OF THE DRAWINGS

FIG. 1 is a schematic representation of chemistry for attachment of an antibody to a hydrophilic molecule extending from a single walled carbon nanotube (SWNT);

FIG. 2 (top) is another representation of chemistry for attachment of an antibody to an (SWNT); (bottom left box) AFM images of the SWNT labels before (left) and after (right) GαM (goat anti-mouse) antibody conjugation; and (bottom right): diagram of label comprising nanotube, PL-PEG (4500)—linker and antibody.

FIG. 3 shows (3A) Raman peak intensity (G-band) on eighteen protein spots (plus 1 control) demonstrating highly selective recognitions of all six different mouse IgGs on the substrate by SWNT-tagged goat anti-mouse (GαM) secondary antibodies; (3B) is a schematic representation of the direct detection. (3C) To verify that the selectivity is due to the protein conjugated to the SWNTs, that protein (Gα M) is identified with another fluorescently tagged antibodies (cy3-conjugated donkey anti-goat); 3D shows florescent results of (3C) showing that only the six positive protein spots (top row) have fluorescent signals, confirming the co-localization of the conjugated proteins and the SWNTs; the proteins are identified as under the heading “Proteins,” e.g., mouse anti-TNF, mouse anti-human serum albumen, etc.

FIG. 4 is a graph showing Raman shift from an SWNT (left) and density of electronic states (right);

FIG. 5 is a series of graphs showing indirect detection results, where 5A is a schematic representation of indirect detection. Various concentrations of M α HSA (52) were exposed to HSA (54) spots and subsequently detected with GαM secondary antibody (56) attached to an SWNT (58): 5(B) shows Raman detection results with increasing concentrations of MαHSA; (5C) shows fluorescence detection of MαHSA using cy3-labeled GαM secondary antibody (instead of SWNT-labeled). Note that the high laser gain needed to see the low concentrations causes the 10-nM spot to saturate. The detection limit (arrow in 5B) of MαHSA by the SWNT label is ˜10 fM compared to ˜10 pM by the cy3 label;

FIG. 6 is a Raman scan in which scan of the same location after 2-h continuous exposure to the Raman laser (as well as ambient light) shows no appreciable degradation of the Raman signal, demonstrating its stability during data acquisition;

FIG. 7 is a 1-μm resolution intensity map of SWNT-labeled GαM over a ˜50-um spot of MαHAS printed by a robotic microarrayer. This Raman image demonstrates usage compatibility of the SWNT-labels with microarray technology. Additionally, details within the spot are better resolved at this resolution than the typical 5-μm limit of the fluorescent scanner used to image microarray slides;

FIG. 8 is a schematic diagram of an SWNT solubilized with an FITC-PEG conjugate (A); and a representation of FITC as shown in (A) showing the planar ring structure (B);

FIG. 9(A) is a schematic of near infrared (NIR) photoluminescence detection of SWNT-anti CD 20 monoclonal antibody conjugate selectively bound to CD 20 cell surface receptors on a B cell lymphoma, while a T cell lymphoma (with no CD 20) does not bind the complex; 9(B) is an NIR photoluminescence spectrum of SWNT-anti CD 20 conjugate, showing typical SWNT emission peaks; and FIG. 9(C) is a photoluminescence spectrum recorded on an SWNT-anti Cd 20 treated Raji cell; and

FIG. 10 (A) shows mean autofluorescence values for a variety of different cell lines (B cell, T cell, breast cancer cells, and epidermoid cancer lines respectively). The values were observed to be low and did not vary much from line to line. This is important when performing high resolution imaging and for best signal to noise ratio; 10(B) shows control experiment comparing binding of SWNT-Rituxan conjugate to negative (CEM) cells to NSB of PEGylated SWNTs to both positive (Raji) and negative cells. The first bar shows the mean fluorescence signal from CEM cells treated with the SWNT-Rituxan conjugate above. The second and third bars show mean fluorescence from Raji and CEM cells incubated with PEGylated SWNTs without the Rituxan antibody. Inset shows a close up of the control samples.

### DETAILED DESCRIPTION OF THE PREFERRED EMBODIMENT

### Definitions

The term “carbon nanoparticle” means a carbon particle having a nominal diameter less than about 100 nm and comprising primarily sp² bonded carbon atoms. Examples include multi-walled carbon nanotubes, fullerenes, and, especially, single walled carbon nanotubes. Carbon nanoparticles can be all carbon (such as the so-called carbon onions), or carbon-coated particles consisting of carbon layers wrapped around other materials, usually carbides. An example of the present nanoparticles (“carbon onions”) and related materials and their preparation is given in Sano et al. “Properties of Carbon Onions Produced by an Arc Discharge in Water,” J. App. Phys. 92(5):2783-2788 (2002).

The term “carbon nanotube” means a tube that contains a sheet of graphene rolled into a cylinder as small as 1 nm in diameter. Both single-walled nanotubes (SWNTs) and multiwalled nanotubes (MWNTs), with many concentric shells, have been synthesized. The electronic properties of a nanotube depend on the angle (chirality) with which it is rolled up—the present nanotubes can be metals, small-gap semiconductors, or large-gap semiconductors. Carbon nanotubes may include other materials. Metallic tubes have shown ballistic conduction on length scales of a micron or more. Nanotubes are also the stiffest known material, with a Young's modulus of ˜1 TPa, which makes them excellent candidates for nanomechanical systems. Carbon nanotubes, as used herein, includes structures that are not entirely carbon, such as BCN nanotubes. The present carbon nanotubes may also be graphene in other forms. This includes a single sheet of graphene formed into a sphere, which constitutes a carbon nanosphere, commonly referred to as a buckyball or fullerene. The present nanotubes may contain other atoms or dopants, such as silicon, etc.

The present carbon nanotubes will have, in contrast to the Raman spectra of the other forms of carbon, which contain at most 3 bands in the fingerprint region of the spectrum (100-1800 cm-1), many bands that are quite sharp, particularly the G band.

In addition to SWNTs and MWNTs, the present carbon nanoparticles may be double walled nanotubes (DWNTs). As described by Colomer et al., “Bundles of identical double-walled carbon nanotubes,” Chem. Commun., 2004, 2592-2593 CCVD may be used to produce such materials. In practice, there may be a range of about 2-5 walls in DWNTs. With DWNTs and MWNTs, the outer walls may be covalently linked to the solubilizing molecule without regard to disruption of sp2 bonding or other aspects of Raman signature, since the signature may be provided by the inner wall(s). A DWNT is a particular type of MWNT, which has predominantly 2 walls.

The term “amphiphilic molecule” is used in its conventional sense, i.e., a chemical compound possessing both hydrophilic and hydrophobic nature. Polar lipids, including phospholipids, (defined below) are such compounds. The lipid, or aliphatic (defined below) portion provides a hydrophobic portion. The hydrophilic portion may be provided by a hydrophilic polymer such as PEG or related compounds. A hydrophilic or amphiphilic molecule is referred to here as a “solubilizing molecule” as it is sufficiently hydrophilic to form a solution or stable suspension comprising the present nanoparticles. As used herein, an amphiphilic molecule is attached at one end to a nanoparticle, which particle is hydrophobic, and at the other end to a hydrophilic portion to provide a solubilizing molecule to enable the composite to be stably suspended in an aqueous environment. The hydrophobic portion of the amphiphilic molecule simply serves as a linker to the nanoparticles, and, instead of a lipid or aliphatic portion, may be pyrene or other polyaromatic molecule. For example, Nakashima et al., “Water-Soluble Single-Walled Carbon Nanotubes via Noncovalent Sidewall-Functionalization with a Pyrene-Carrying Ammonium Ion,” Chem. Lett., Vol. 31 (2002), No. 6 p. 638, describes a process in which sonication of solid single-walled carbon nanotubes (p-SWNT) in an aqueous solution of a pyrene-carrying ammonium ion gave a transparent dispersion/solution of the nanotubes. As described in US 20060189822 to Yoon et al., published Aug. 24, 2006, entitled “Dispersant for dispersing carbon nanotubes and carbon nanotube composition comprising the same,” the present amphiliphic molecules may comprise a hydrophobic portion selected from a variety of aromatic compounds such as benzene, and polycyclic aromatic hydrocarbon groups, including naphthalene, imidazole, acenaphthalene, acenaphthene, fluorene, anthracene, phenanthrene, fluoranthene, pyrene, benzanthracene, chrysene, benzo(b)fluoranthene, benzo(k)fluoranthene, benzopyrene, benzoperylene, indeno(1,2,3-c,d)pyrene, etc. As further disclosed there, the hydrophilic polymer may be polyethylene oxide or polypropylene oxide, or polymethylmethacrylate, polybutylmethacrylate, polyacrylic acid, polymethacrylic acid, polyoxyethylene, polyoxypropylene, or copolymers of polyalkylmethacrylate and polymethacrylic acid. Pyrene adsorption onto a nanotube is also described in Acc. Chem. Res. 2002, 35, 1035-1044.

The term “crosslinker” is used herein in its conventional sense, i.e., a molecule that can form a three-dimensional network when reacted with the appropriate base monomers.

The term “polar lipid” refers to a molecule having an aliphatic carbon chain with a terminal polar group. Preferred polar lipids include but are not limited to acyl carnitine, acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid. Further polar lipids are exemplified in U.S. Pat. No. 6,339,060, “Conjugate of biologically active compound and polar lipid conjugated to a microparticle for biological targeting,” to Yatvin, et al., hereby incorporated by reference.

The term “phospholipid” means a molecule having an aliphatic carbon chain with a terminal phosphate group. Typically the phospholipids will comprise a glycerol backbone, attached to two fatty acid (aliphatic groups) esters and an alkyl phosphate. Suitable phospholipids for use in this invention include, without limitation, dimyristoyl phosphatidylcholine, distearoyl phosphatidylcholine, dilinoleoyl-phosphatidylcholine (DLL-PC), dipalmitoyl-phosphatidylcholine (DPPC), soy phophatidylchloine (Soy-PC or PCs) and egg phosphatidycholine (Egg-PC or PCE). Suitable phospholipids also include, without limitation, dipalmitoyl phosphatidylcholine, phosphatidyl choline, or a mixture thereof. Exemplified below are 1,2-dipalmitoyl-sn-glycero-3 phosphoethanolamine phospholipid and 1,2-Distearoyl-sn-Glycero-3-Phosphoethanolamine.

The term “aliphatic,” as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl” and the like. Furthermore, as used herein, the terms “alkyl,” “alkenyl,” “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The aliphatic(lipid) alkyl groups employed in the lipids of the invention preferably contain 4-20, more preferably 10-20 aliphatic carbon atoms. In certain other embodiments, the lower alkyl, (including alkenyl, and alkynyl) groups employed in the invention contain 1-10 aliphatic carbon atoms. Illustrative aliphatic groups thus include, but are not limited to, for example, methyl, ethyl, n-propyl, isopropyl, cyclopropyl, —CH₂-Cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like. The aliphatic groups are hydrophobic and adsorb to the hydrophobic nanoparticle.

The term “PEG” means polyethylene glycol, a polymer with the structure (—CH₂CH₂O—)ₙ that is synthesized normally by ring opening polymerization of ethylene oxide. The PEG used herein will impart water (and serum) solubility to the hydrophobic nanoparticle and lipid portion of the polar lipid. The polymer is usually linear at molecular weights (MWs)≦10 kD. The PEG used here will have an MW around 5,400, preferably above 2,000, or about 45 repeating ethylene oxide units. However, the higher MW PEGs (higher “n” repeating units) may have some degree of branching. Polyethylene glycols of different MWs have already been used in pharmaceutical products for different reasons (e.g., increase in solubility of drugs). Therefore, from the regulatory standpoint, they are very attractive for further development as drug or protein carriers. The PEG used here should be attached to the nanoparticles at a density adjusted for the PEG length. For example, with PL-PEG 2000, we have an estimate of ˜4 nm spacing between PEG chain along the tube.

For coupling proteins to PEG, usually monomethoxy PEG [CH₃(—O—CH₂—CH₂)ₙ—OH] may be first activated by means of cyanuric chloride, 1,1′-carbonyldiimidazole, phenylchloroformate, or succidinimidyl active ester before the addition of the protein. In most cases, the activating agent acts as a linker between PEG and the protein, and several PEG molecules may be attached to one molecule of protein. The pharmacokinetics and pharmacodynamics of the present nanotubes-PEG-protein conjugates are expected to be somewhat dependent on the MW of the PEG used for conjugation. Generally the presently used PEG will have a molecular weight of approximately 4,000-10,000 Daltons.

The present PEG may also modified PEG such as PolyPEG® (Warwick Effect Polymers, Ltd., Coventry, England) is new range of materials suitable for the attachment of polyethylene glycol (PEG) to therapeutic proteins or small molecules. These are prepared using Warwick Effect Polymers' polymerization technology, (See U.S. Pat. No. 6,310,149) and contain terminal groups suitable for conjugation with, among other things, lysine, terminal amino and cysteine residues.

The term “hydrophilic polymer” means a polymer with a solubility of at least ten grams/liter of an aqueous solution at a temperature of between about 0 and 50° C. Aqueous solutions can include small amounts of water-soluble organic solvents, such as dimethylsulfoxide, dimethylformamide, alcohols, acetone, and/or glymes.

Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll® polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

The term “polypeptide” or “protein” means a polymer of amino acids without regard to the length of the polymer, provided that the protein has specific binding properties. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1 12, 1983; Seifter et al., Meth Enzymol 182:626 646, 1990; Rattan et al., Ann NY Acad Sci 663:48 62, 1992). Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “antibody” means any of several classes of structurally related proteins, also known as immunoglobulins, that function as part of the immune response of an animal, which proteins include IgG, IgD, IgE, IgA, IgM and related proteins which specifically bind to their cognate antigens. This term further relates to chimeric immunoglobulins, which are the expression products of fused genes derived from different species. These terms further relate to immunologically active derivatives of the above proteins, including, but not limited to, an F(ab′)2 fragment, an Fab fragment, an Fv fragment, a heavy chain, a light chain, an unassociated mixture of a heavy chain and a light chain, a heterodimer consisting of a heavy chain and a light chain, a catalytic domain of a heavy chain, a catalytic domain of a light chain, a variable fragment of a light chain, a variable fragment of a heavy chain, and a single chain variant of the antibody. The term “antibody” here refers to an antibody of a single specificity rather than a mixed population of antibodies.

The term “specific binding” means that binding which occurs between such paired species as enzyme/substrate, receptor/agonist or antagonist, antibody/antigen, complementary polynucleotides and lectin/carbohydrate which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specific binding” occurs between a paired species where there is interaction between the two, which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “ligand” means all molecules capable of specifically binding to a particular target molecule and forming a bound complex as described above. Thus for example, an antibody against mouse IgG specifically binds the IgG ligand.

The term “polynucleotide” means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g., naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Polynucleotides typically range in size from a few monomeric units, e.g., 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right. “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. The present polynucleotides may also comprise non-natural nucleotide analogs, e.g., including modified bases, sugars, or internucleosidic linkages. It is clear to those skilled in the art that where an enzyme has specific oligonucleotide or polynucleotide substrate requirements for activity, e.g., single-stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

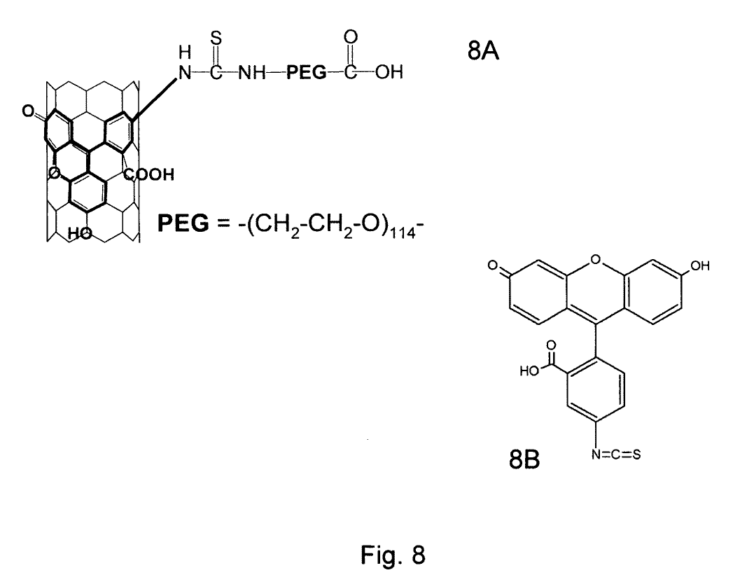

The term “fluorophore” means a molecule or a component of a molecule that causes a molecule to be fluorescent. The term is used here particularly to refer to small, hydrophobic molecules such as luciferin, quantum dots, and fluorescein. In certain embodiments, the present fluorophores will comprise a fluorescein derivative, as that term is customarily used. Fluorescein derivatives generally contain the tricyclic and benzyl groups shown in FIG. 8B, but will have different amino-reactive groups used for coupling the fluorophore. Certain ring substitutions can also be made, which will change fluorescence properties. Such derivatives include FITC (fluorescein-5-isothiocyanate), available from Molecular Probes, Pierce, and other suppliers; DTAF (Dichlorotriazinylaminofluorescein), available from Molecular Probes, Pierce, and other suppliers; and NHS-Fluorescein, available from Molecular Probes, Pierce, and other suppliers. Various isomers of FITC are included in the present definition, such as 5-((5-aminopentyl)thioureidyl)fluorescein, 6-FITC, fluorescein-6-isothiocyanate, etc.

The term “biological labeling molecule” means a biological molecule that is attached to the nanoparticle complex for specific labeling of an analyte. That is, the labeling molecule may be a polynucleotide or protein, such as an antibody, which acts as a ligand to, and exhibits specific binding to, an analyte. The analyte may be free in a sample, or may be in a tissue or on a cell to be labeled.

### Overview

Our approach for making the SWNT-labeled biomolecules is to first solubilize the SWNTs in aqueous solutions using a surfactant (or solubilizing molecule) with the following criteria: 1) An amphiphile whose hydrophilic domain contains sufficient biomolecule-repelling groups, to allow aqueous solubility and preventing nonspecific binding of SWNTs. 2) Available linkable functional groups. Cross-linkers and/or activators are then used to conjugate desired biomolecules to the above-solubilized SWNTs. The resulting SWNT-labeled biomolecules can be used to detect the complement of the conjugated biomolecule by measuring the Raman signal of the SWNT-label.

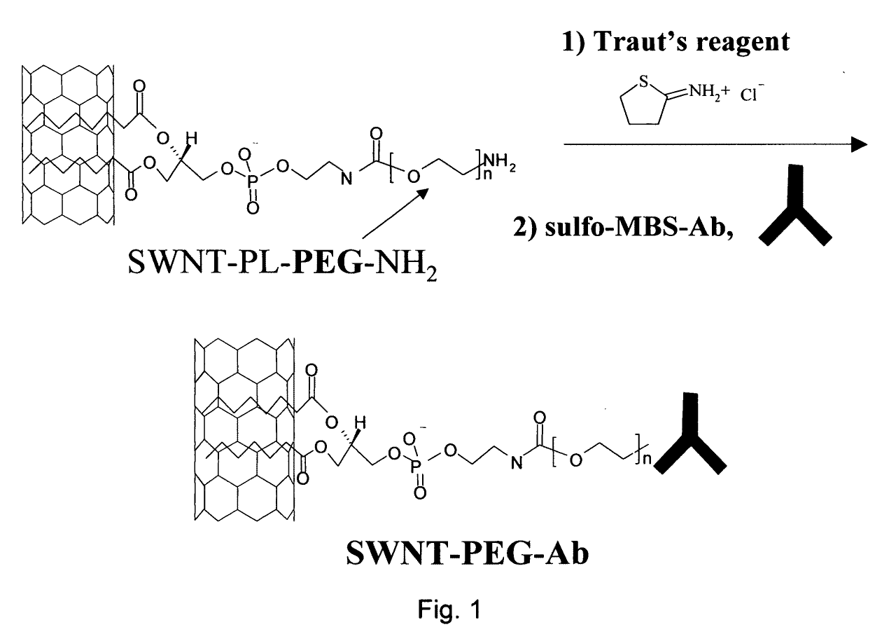

Two conjugation schemes have been employed in the present method. FIG. 1 shows the first scheme. As can be seen on the left, a phospholipid is adsorbed on an SWNT through the lipid portion. The phosphate head group is linked through an amide bond to PEG, with the repeating ethylene units shown. The PEG terminates in an amine group, which is thiolated using a cross linker such as Traut's reagent (2-Iminothiolane.HCl), available from Pierce chemical. Traut's reagent reacts with amine groups leaving a free thiol group. A sulfhydryl reactive cross linker such as sulfo-MBS (m-Maleimidobenzoyl-N-hydroxysulfosuccinimide ester) which contains the reactive groups sulfo-NHS ester and maleimide, reactive with amino and sulfhydryl groups, is added to the protein. The sulfo-NHS ester group of sulfo-MBS modifies primary amines on the protein, located at the N-terminus and on lysine side chains, and the other end of the crosslinker then binds with the thiolated SWNT.

FIG. 1 illustrates PEG (5400) NH₂, but other PEG species may be used.

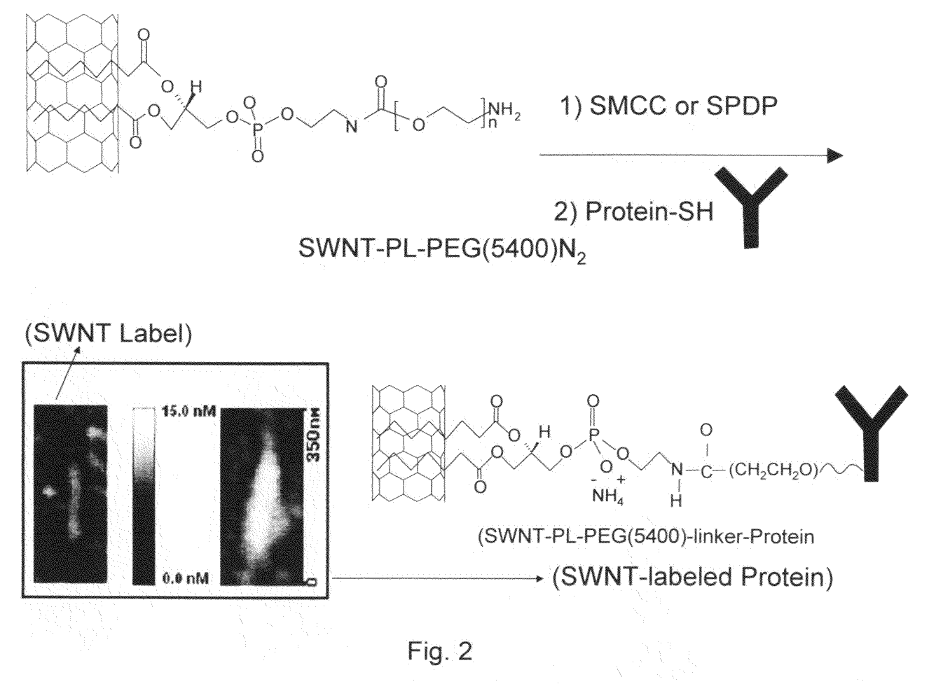

FIG. 2 shows the second scheme. In this case, the amine group at the end of PEG on the SWNT is linked to a cross linker such as Sulfo-LC-SPDP and sulfo-SMCC, available from Pierce chemical, which contain an amino reactive group (sulfo-NHS ester) and a thiol reactive group (either disulfide or maleimide). After this, the other end of the crosslinker is bound to a thiolated protein, shown in FIG. 2 as a “Y” for antibody. The protein is thiolated using Traut's reagent. The protein to be labeled is exemplified by an antibody, e.g., a goat anti-mouse antibody (GαM).

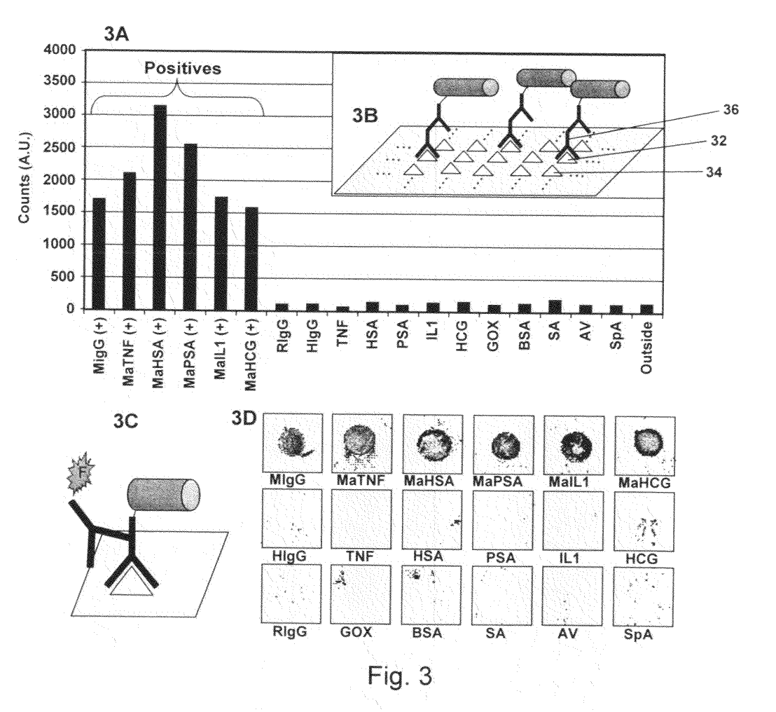

A successful implementation of this invention has been demonstrated by the use of PEG at different MWs, including MW 5400 amine (PL-PEG(5400)NH₂) (Avanti Polar Lipids), which was used to solubilize and functionalize the SWNTs with goat anti-mouse secondary antibodies (GαM). Two different cross-linkers (sulfo-LC-N-Succinimidyl 3-(2-pyridyldithio) propionate (SPDP) and 4-(N-Maleimidomethyl)cyclohexane-1-carboxylic acid 3-sulfo-N-hydroxysuccinimide ester sodium salt (SMCC)) have been successfully used to attach thiolated GαM (via Traut's reagent) to the PL-PEG(5400)NH2-solubilized SWNTs. The selectivity of these SWNT-labeled GαM has been shown by measuring the G-band, Raman signature of the SWNT (1600 cm-1) on spotted proteins on solid substrate (results using SPDP are shown in FIG. 3). Results indicate the detection limit to be 10 fM, in contrast to 10 pM by fluorescence. The present methods can be adapted for improving the limit of detection, performing sensing in complex biological backgrounds, demonstrating successful detections with clinical samples, and shelf-life determination.

As-produced SWNTs are highly hydrophobic, but were stably dispersed into aqueous solution for use as biological labels. This was accomplished by sonicating SWNTs with pegylated (MW 2000) phospholipids, which have been shown to be excellent solubilizers of SWNTs. This process also cuts the nanotubes to much more uniform lengths (142.2±61.9 nm, by AFM). Additionally, high-speed centrifugation of this suspension offers a way to remove impurities from the growth process as sedimented pellet. While this supernatant makes a stable solution of SWNTs, we also found that additional PEG units (MW 3400) were useful to fully eliminate nonspecific binding on the substrate, yielding a final PEG MW of about 5400.

An AFM image of the resulting SWNT label is shown in FIG. 2. Topographic analysis indicates the diameter of the nanotubes to be ˜3-4 nm; with the added height from the average, 1.1-nm as-grown SWNT representing the PEG units.

As shown below, a protective PEG layer on the SWNT label permitted simultaneous, selective sensing to be achieved. The ˜10 fM detection limit outperforms traditional fluorescent technique by 3 orders of magnitude. Additionally, the SWNT labels can be used in complex biological background with time-stable signal and minimum sample volume requirement, and are compatible with microarray technology. As such, it is an attractive replacement of fluorescent dye, and motivates the development of a high-speed Raman scanner for future high-throughput capability.

Raman spectroscopy or surface plasmon resonance may be used here for sensitive and accurate detection or identification of individual molecules from biological samples, as described in US 2005/0148100. When light passes through a medium of interest, a certain amount of the light becomes diverted from its original direction, which is known as scattering. Some of the scattered light also differs in frequency from the original excitatory light, due to the absorption of light and excitation of electrons to a higher energy state, followed by light emission at a different wavelength. The difference of the energy of the absorbed light and the energy of the emitted light matches the vibrational energy of the medium This phenomenon is known as Raman scattering, and the method to characterize and analyze the medium or molecule of interest with the Raman scattered light is called Raman spectroscopy. The wavelengths of the Raman emission spectrum are characteristic of the chemical composition and structure of the Raman scattering molecules in a sample, while the intensity of Raman scattered light is dependent on the concentration of molecules in the sample. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity. A Raman label may be used to generate a signal having increased intensity.

Both rotational and vibrational Raman spectroscopy are possible. The energy of the exciting radiation will determine which type of transition occurs—rotational transitions are lower in energy than vibrational transitions. In addition to this, rotational transitions are around 3 orders of magnitude slower than vibrational transitions. Therefore, collisions with other molecules may occur in the time in which the transition is occurring. A collision is likely to change the rotational state of the molecule, and so the definition of the spectrum obtained will be destroyed. Rotational spectroscopy is therefore carried out on gases at low pressure to ensure that the time between collisions is greater than the time for a transition.

The basic set-up of a Raman spectrometer involves a light source (laser), a sample and a detector orthogonal to the sample. In the present method, the source is preferably an array of samples, which are to be assessed for binding of a label to the sample. The samples will contain biological molecules, which do not themselves generate Raman signals. For a molecule to be active in Raman spectroscopy, it must be polarizable. The oscillating electric field of a photon causes charged particles (electrons and, to a lesser extent, nuclei) in the molecule to oscillate. This leads to an induced electric dipole moment.

The single-walled carbon nanotube, with its unique spectroscopic Raman signature, was utilized here as a novel biological label due to its strong resonant Raman signal that allows for highly sensitive detection. Among their signature peaks, the G-band at ˜1605 cm-1, characteristics of the E2g stretching mode of graphite, is typically highest in intensity, and as such, is the signal followed.

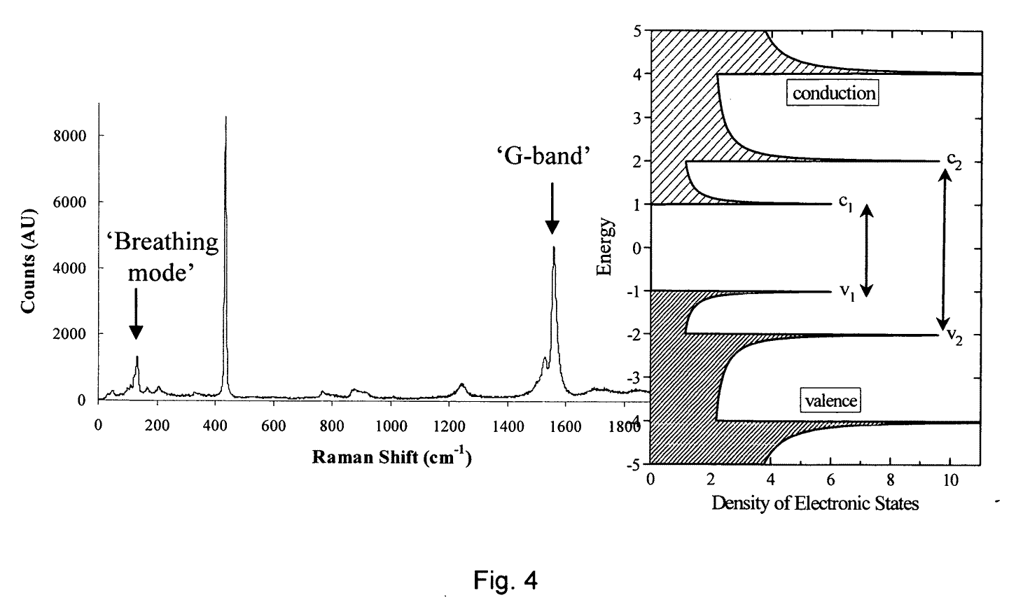

As shown in FIG. 4, the Raman spectrum of an SWNT shows peaks in several areas. Each part of the Raman spectrum, the radial breathing mode (RBM), the disorder-induced mode (D mode between about 1300 and 1400 cm-1) and the high-energy mode (HEM), can be used to access different properties of single-walled carbon nanotubes as labels. In the RBM mode the present detecting will be in the range of 0-400 cm-1, as shown in FIG. 4. In the high-energy range around 1600 cm-1 single-walled nanotube show a characteristic double-peak structure, shown as “G-band” in FIG. 4. The frequency of the radial breathing mode is proportional to the inverse of the nanotube diameter. The diameter of carbon nanotubes can be estimated by measuring the RBM frequency. In metallic carbon nanotubes the lower high-energy mode is strongly broadened and shifted to smaller energies (1540 cm-1). This so-called metallic spectrum appears only in metallic tubes and for a properly chosen energy of the incoming laser light. Double-resonant Raman scattering occurs in carbon nanotubes and other sp2 carbon over the entire visible energy range. This effect arises from the peculiar electronic band structure of single-walled nanotubes and graphite.

The present methods are exemplified by resonant Raman scattering, although other techniques may be used, such as Surface Enhanced Raman Scattering (SERS). For example, U.S. Pat. No. 7,019,828 to Su, et al., issued Mar. 28, 2006, entitled “Chemical enhancement in surface enhanced Raman scattering using lithium salts,” describes a suitable system for surface-enhanced Raman spectroscopy (SERS).

The current detection limit is also highly competitive with other nanoparticle-based techniques, including Raman-active dye on Au nanoparticles (20 fM), even without an amplification step. Kneipp et al (Kneipp, K.; Kneipp, H.; Dresselhaus, M. S.; Lefrant, S., Surface-enhanced Raman scattering on single-wall carbon nanotubes. Philosophical Transactions of the Royal Society London, Series A (Mathematical, Physical and Engineering Sciences) 2004, 362, (1824), 2361) however reported that the effect of surface-enhanced Raman scattering (SERS) of SWNTs can increase the signal up to 14 orders of magnitude when the nanotubes are in contact with silver or gold nanostructures. Indeed, we observed signal amplification after an evaporation of a thin Au film. It is expected that optimizing the detection sensitivity will push the detection limit passed the attomolar level achieved by combined nanoparticles methods. (See Jwa-Min, N.; Thaxton, C. S.; Mirkin, C. A., “Nanoparticle-based bio-bar codes for the ultrasensitive detection of proteins,” Science 2003, 301, (5641), 1884.) Applications of this technique in detecting low-abundance serum proteins are also being demonstrated. Lastly, this technique also has the potential for multiplexed detection by utilizing distinctive low frequency (<200 cm-1) radial breathing modes (RBMs) of different diameter SWNTs instead of the collective G-band signature. The combined benefits of these water-suspended SWNT labels make them ideal alternative bio-labels for applications in ultrahigh sensitivity proteomic studies.

Variations in the SWNT-biomolecule production scheme span the different choices of surfactants, linkers/activators, and biomolecules. Possible usage ranges from on-substrate imaging (similar to microarray, tissue staining) to solution phase detection (similar to flow cytommetry/cell sorter) and real-time monitoring of binding events. Raman detection can be performed using other kinds of nanotubes (multi-walled or chemically modified NTs), and can also be multiplexed by using individual unique Raman peaks of different types of CNTs (i.e., diameters and chiralities). Further, the signal can be enhanced with gold/silver nanoparticles.

An alternative method for providing a solubilizing molecule (i.e., providing a stable aqueous suspension without settling out of nanoparticles for at least a day, preferably at least three days) is given in Kam et al., “Carbon nanotubes as multifunctional biological transporters and near-infrared agents for selective cancer cell destruction,” Proc Natl Acad Sci USA, 2005 Aug. 16; 102(33): 11600-11605. As described there, SWNTs may be functionalized by various phospholipids (PL) with a polyethylene glycol (PEG). PL with a PEG moiety and fluorescein tag (PL-PEG-FITC) was reported in this paper. Three milligrams of PL-PEG-NH₂ was dissolved in 1.5 ml of 0.1 M carbonate buffer solution (pH 8.0). To this solution 100 μl of 13 mM solution of FITC in DMSO (Aldrich) was added. The mixture was allowed to react overnight at room temperature and protected from light. Purification by gel chromatography was achieved by loading 1 ml of the solution to a Sephadex G-25 column (Aldrich). As elution solvent (H₂O) was flown through the column the formation of two separate yellow bands was observed. The fractions were collected, and the absorbance of various fractions was measured at 488 nm with a HP-8453 spectrophotometer. Fractions from the first elution peak were pooled as they were attributed to the higher molecular weight PL-PEG-FITC conjugate (also confirmed by fluorescence measurement), and subsequently used for solubilization of SWNTs.

As another alternative, the solubilizing molecule may be covalently attached to carbon nanoparticles, eliminating the need for an amphiphilic molecule—particularly the hydrophobic portion. In this case, the carbon nanoparticles is covalently functionalized with a linking group, and the hydrophilic group is attached to the linking group. For example, an acid group may be covalently bound to a carbon nanotube surface, and an amino-PEG linked thereto. Chemistry for functionalizing carbon nanotubes (applicable to SWNT, DWNT, i.e., double-walled nanotubes, and MWNT, i.e., multi-walled nanotubes) is exemplified in US 20060199770 to Bianco, et al., published Sep. 7, 2006, entitled “Functionalized carbon nanotubes, a process for preparing the same and their use in medicinal chemistry.” Another method to covalently link molecules to an SWNT is described in Saini et al., “Covalent sidewall functionalization of single wall carbon nanotube,” J Am Chem Soc., 2003 Mar. 26; 125(12):3617-21. In that method, alkyl lithium reagents were used to attach alkyl groups to the sidewalls of fluoro nanotubes.

The present solubilized carbon nanoparticle conjugates, as described above, can also be used as specific probes for cell surface receptors, using specific biological labeling molecules, such as antibodies. Described below are examples using commercially available, clinically approved monoclonal antibodies, which were thiolated with Traut's reagent. Traut's reagent is further described in U.S. Pat. No. 6,428,579. The thiolated antibodies are then coupled to amine groups on NH2-PEG. The PEG is attached to a phospholipid, which is adsorbed on the hydrophobic carbon surface of the nanoparticle. The carbon nanoparticles used were Hipco produced and sized to be about 83 nm in length and about 1.6 nm after PEGylation. NIR fluorescence images were obtained showing labeling of specific cells. While the present work utilizes an inverted fluorescence microscope, NIR spectroscopes are now available commercially. Basically these consist of pulsed laser diodes generating near infrared light of different wavelengths (between 7-1300 nm wave lengths) carried to the patient via a fiber optic cable (optode). Scattered light is collected by a second optode and detected by a photomultiplier tube. NIR spectroscopy can be carried out in vivo. See, Matter, et al., “Molecular imaging of atherosclerotic plaques using a human antibody against the extra-domain B of fibronectin,” 1: Circ Res. 2004 Dec. 10; 95(12):1225-33. Epub 2004 Nov. 11. There, recombinant miniantibody (SIP) format of L19 was used to image plaques in atherosclerotic mice using antibody labeling with radioiodine or near-infrared fluorophores and evaluated the expression of fibronectin ED-B in murine and human plaques. Macroscopic near-infrared imaging was performed using a home-built infrared fluorescence imager and a CCD camera (C5985; Hamamatsu; 800×600 pixels, spatial resolution 125 μm), with exposure times between 0.5 and 2 seconds. 26 Signal/noise ratio in fluorescence images of longitudinally opened aortas was determined using linear integration of signal intensity (NIH Image 1.63f software) over corresponding areas of aortas, taking the mean of 3 measurements. Using the methods described below, one could perform in vivo imaging of cells using the described carbon nanoparticle complexes.

The present NIR techniques may also be used for in vitro cell labeling. Since an IR laser is used, Raman scattering may be measured at the same time as IR absorbance is measured for purposes of spectroscopy. As described in U.S. Pat. No. 5,481,113 to Dou, et al., issued Jan. 2, 1996, entitled “Apparatus and method for measuring concentrations of components with light scattering,” when Raman scattered light is used for measurement of concentrations of components, intense fluorescence generated by the measuring object causes a great background signal to be issued, which would make an obstacle to correct concentration measurement. However, by using laser in near-infrared wavelength ranges at low quantum energy level, occurrence of fluorescence can be avoided, so that the S/N ratio is improved and therefore measurement accuracy can also be improved.

### METHODS AND MATERIALS

### Proteins:

The sources of proteins used were as follows: Prostate Specific Antigen (PSA), Human Chrionic Gonadotropin (HCG), mouse anti-PSA, mouse anti-HCG, human IgG (HIgG), Goat anti-Mouse secondary antibodies (GαM) (BiosPacific); Tumor Necrosis Factor alpha (TNF), Interleukin-1 beta (IL-1), mouse anti-TNF, mouse anti-IL1 (PeproTech); Protein A (SpA) (Pierce Biotechnology); streptavidin (SA), avidin (Av), glucose oxidase (GOX), bovine serum albumin (BSA), human serum albumin (HSA), mouse anti-HSA, rabbit IgG (RIgG), mouse IgG (MIgG) (Sigma-Aldrich); cy3-conjugated donkey anti-goat, cy3-conjugated goat anti-mouse secondary antibodies, fetal calf serum (FCS) (Invitrogen).

### Other Materials:

High-Pressure Carbon Monoxide (HiPCO)-produced SWNT was provided by Carbon Nanotechnologies. Phospholipids-PEG(2000)-NH2 (1,2-Distearoyl-sn-Glycero-3-Phosphoethanol amine-N-[Amino(Poly ethylene Glycol)2000]Ammonium Salt) was purchased from Avanti Polar Lipids; NHS-PEG(3400)-BOC from (Nektar Therapeutics); Sulfo-LC-SPDP (Sulfosuccinimidyl 6(3-[2-pyridyldithio]-propionamido)hexanoate) from Piece Biotechnology; Phosphate Buffered Saline, pH 7.4 (PBS) from Invitrogen; SMCC (Succinimidyl 4-[N-maleimidomethyl]cyclohexane-1-carboxylate) and Tween 20 from Sigma-Aldrich. All proteins and chemicals were used without further purification.

Dialysis membranes (Spectra/Por Biotech Cellulose Ester) were obtained from SpectrumLabs. Centrifugal filter devices were purchased from Ultracon. ProteinReady slides were acquired from Advansta (Menlo Park, Calif.).

### Instrumentation

Cole-Parmer's ultrasonicator cleaner (B3-R); Sorvall Legend's centrifuge (MACH 1.6R); and Varian's UV-Vis-NIR Spectrometer (Cary 6000i) were used in the SWNT-labeling process. Atomic Force Microscopy (AFM) images were obtained with a Digital Instrument's Nanoscope IIIa AFM. A robotic microarrayer using ChipMaker 2 Micro Spotting Pins (TeleChem International) was used to spot protein microarray slides. Fluorescent scans were carried out with Axon's GenePix 4000B microarray scanner.

Raman data were obtained using a Renishaw Raman microscope with Leica DM/LM stage, using 785 nm laser (with ˜1 μM spot size). Each scan was set to accumulate for 30 s; and an average of 5 different locations in each protein spots was reported. It is contemplated here that laser excitation wavelengths of 400 nm to 2 microns can be used.

### Example 1

### Production of SWNT-PEG Labels

SWNT labels were obtained from solubilizing HiPCO-produced SWNTs with Phospholipids-PEG(2000)-NH221 by sonicating 0.25 mg/ml of the nanotube with 1 mg/ml of the phospholipids in water for 1 h. Large aggregates and impurities from the HiPCO process were then removed by high-speed centrifugation (25000 g, for 6 h), and the supernatant retained. Excess Phospholipids-PEG(2000)-NH2 was filtered away using Ultracon's 100k MWCO centrifugal device (until the retentate was no longer bubbly).

Additional PEG units were added to the initial PEG chain to further reduce nonspecific binding of the SWNT labels on substrate. 1 mM of NHS-PEG (3400)-BOC was allowed to react with the SWNT-PLPEG2000-NH2 above in 0.1 M phosphate buffer (pH 7.5) at RT for 2 h. After excess reagents was removed with Ultracon's 100k MWCO centrifugal device, the BOC groups were deprotected to amines by heating overnight at 80° C., resulting in a stable, black solution of SWNT labels ready for conjugation with proteins.

In one representative protocol, 34 mg of NHS-PEG-BOC-3400 (purchased from Nektar Therapeutics) and 10 mg of PL-PEG-NH₂ (1,2-Distearoyl-sn-Glycero-3-Phosphoethanolamine-N-[Amino(Polyethylene Glycol)2000] purchased from Avanti Polar Lipids) were dissolved in 2.5 mL of dried methanol. 100 mM of N-Ethyl-N′-(3-dimethylaminopropyl)carbodiimide (EDC, purchased from Aldrich) and 5 mM of N-hydroxysuccinimide (NHS) ester (purchased from Aldrich) were added to the mixture and allowed to react for ˜16 hours. The methanol was then evaporated by blow-drying the solution with air. The BOC protecting group was then deprotected with 2.5 mL of 100% trifluoroacetic acid for ˜16 hrs. The solution was the diluted to ˜15 mLs, and the acid and excess reagents were filtered using 5000 Da molecular cutoff centrifugal device (Amicon Ultra, Millipore). The resulting PL-PEG(5400)-NH₂ retentate was washed by adding water and filtering until the PL-PEG(5400)-NH₂ solution was no longer acidic. PL-PEG(5400)-NH₂ was resuspended at a concentration of ˜360 μM (10 mL). This solution was sonicated with 0.25 mg/mL single-walled carbon nanotubes (SWNTs, as produced HiPco purchased from Carbon Nanotechnologies) for ˜1 hr and centrifuged at ˜25,000 g for 6 hrs. The pellet formed at the bottom of the centrifuge tube containing aggregates, bundles, and impurities was discarded, and the supernatant was collected and filtered through a centrifugal filter device (Millipore Amicon 100k Da molecular cutoff) to remove excess material and resuspended in 0.1 M phosphate buffer at pH 7.5. The SWNT-PL-PEG suspension was characterized by UV-visible-NIR spectroscopy (Cary 6000i) to determine the concentration (ε=7.9×106 M-1/cm-1 at 808 nm).

### Example 2

### Labeling of Proteins with SWNTs

Protein conjugation was accomplished using heterobifunctional cross-linkers to attach the primary amines on the SWNT labels to thiol groups on (thiolated) proteins. Specifically, the SWNT labels were activated with 1 mM of the cross-linker SMCC or SPDP for 2 h in 0.1 M phosphate buffer (pH 7.5) at RT before excess cross-linkers was removed with centrifugal filter (100k MWCO). The retentate were then resuspended in 0.1 M phosphate buffer (pH 6.8) with 5 mM EDTA at 400 nM in concentration (as determined by UV-VIS absorption based on reference 26), and 1 μM of freshly thiolated proteins (via Traut's reagent, excess removed) was then added. The reaction was allowed to proceed for 2 h at RT then overnight at 4° C. before excess proteins was separated by dialysis in PBS with 5 mM EDTA (300k MWCO membrane at 4° C. for 5 days). The SWNT-labeled proteins were kept at 4° C. and used at the concentration of 50 nM (via UV-VIS).

FIG. 2 illustrates the SWNT label conjugation to reporting proteins via heterobifuncational cross-linkers (SMCC or SPDP). The amine units on the SWNT labels are converted to thiol-reactive groups by the cross-linkers and thiolated proteins can then be attached. Based on the stoichiometric ratio of the reaction, there should be 1-2 proteins per nanotube. The AFM image of the conjugate deposited on a silicon substrate in FIG. 2 (bottom) shows AFM images before (left) and after (right) antibody conjugation, showing a topographic height of 13-15 nm along the length of the nanotube, consistent with decorations of IgG molecules (14.2×8.5×3.8 nm28) that are dried.

Antibody-SWNT conjugation was carried out by two representative protocols, using different reagents to activate the SWNT-PEG conjugate for crosslinking to the thiolated protein (goat anti-mouse, GαM).

Scheme 1: 10 mM of Traut's reagent (2-Iminothiolane.HCl, Pierce Biotechnology) was added to 400 μL of 400 nM of SWNT-PL-PEG(5400)-NH₂ in 0.1 M phosphate buffer at pH 7.5 with 5 mM EDTA. After 1 hour, the suspension was filtered using a centrifugal device (Millipore Amicon 100k Da molecular cutoff) to remove excess Traut's reagent and washed 7 times. The resulting SWNT-PL-PEG(5400)-SH retentate was then resuspended in 200 μL 0.1 M phosphate buffer at pH 6.8 with 5 mM EDTA. 800 μM of sulfo-MBS (m-Maleimidobenzoyl-N-hydroxysulfosuccinimide ester) was added to 80 μL of 20 μM of goat-anti mouse IgG (GαM) in 0.1 M phosphate buffer at pH 7.5. After 30 min, the solution was filtered using a centrifugal filter (Millipore Microcon 100k Da molecular cutoff) to remove excess sulfo-MBS and washed 5 times with 0.1 M phosphate buffer at pH 6.8. The activated GαM was resuspended in a small volume (˜50 μL) of 0.1 M phosphate buffer at pH 6.8 and added to 200 μL of 800 nM SWNT-PL-PEG(5400)-SH in 0.1 M phosphate buffer at pH 6.8 with 5 mM EDTA. The activated GαM and SWNT-PL-PEG(5400)-SH were allowed to react for 1 hour at room temperature and then overnight at 4° C. Excess GαM was removed by dialysis for ˜7 days (1 M Da molecular cutoff, SpectrumLabs).

Scheme 2: The SWNT labels were activated with 1 mM of the cross-linker SMCC or SPDP for 2 h in 0.1 M phosphate buffer (pH 7.5) at RT before excess cross-linkers was removed with centrifugal filter (100k MWCO). The retentate were then resuspended in 0.1 M phosphate buffer (pH 6.8) with 5 mM EDTA at 400 nM in concentration (as determined by UV-VIS absorption based on reference 26), and 1 μM of freshly thiolated proteins (via Traut's reagent, excess removed) was then added. The reaction was allowed to proceed for 2 h at RT then overnight at 4° C. before excess proteins was separated by dialysis in PBS with 5 mM EDTA (300k MWCO membrane at 4° C. for 5 days).

### Example 3

### Preparation of Protein Substrates

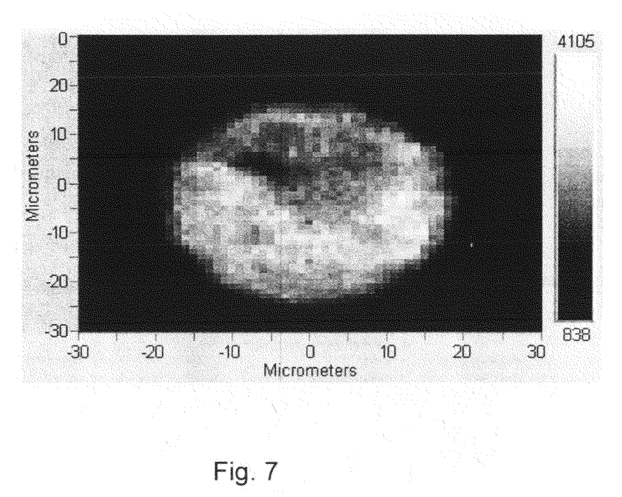

The protein substrates used to test the developed SWNT-labeled protein were generated by spotting 200 nl of 1 uM of each protein on the ProteinReady slides, and allowed to dry (a microarray printer was utilized instead in generating the ˜50 μm spot shown in FIG. 7). The slides were then blocked with 3% FCS 0.1% Tween 20 in PBS overnight at 4° C.; and was rinsed with water and blown dry before subsequent incubation steps.

### Example 4

### Selective Detection by the SWNT-Label Protein

The utility of the SWNT labels is demonstrated with selective detection of surface proteins. By labeling goat anti-mouse secondary antibodies, a common reporting molecule in immunoassays, with the SWNT labels, highly selective recognition of mouse immunoglobulins was obtained (FIGS. 3A and B). In this direct detection, 50 nM of the SWNT-labeled GαM is incubated for 1.5 h, rinsed 3× with PBS for 30 min, squirted with water and blown dry. The Raman intensity from the protein spots shows that all six mouse IgGs (a polyclonal and 5 monoclonals) are correctly identified, whereas the twelve unrelated proteins as well as background area produces minimum signals. Referring now to FIG. 3A, the positive results from MIgG, MaTNF, MaHSA, MaPSA, MaIL1 and MsHCG show counts greater than 1500 AU (arbitrary units), greater than a 5 fold increase for the protein spots treated with antibodies to the indicated proteins, then labeled with SWNT-tagged goat anti-mouse antibodies. The untagged spots were all negative.

As shown in FIG. 3B, triangles 32, 34 represent a number of proteins spotted on the surface. Targeted mouse IgGs 36 are subsequently detected by GαM secondary antibodies (top) labeled with SWNTs. Triangles 32, 34 represent protein spots, which either have a corresponding antibody attached (36) or were not recognized by an antibody (34).

Fluorescent detection was employed to confirm that the selectivity observed is due to the ‘on-tube’ protein (GαM) (FIG. 3C). When the array from FIG. 3A was incubated with 50 nM of cy3-conjugated donkey anti-goat secondary antibodies for 1.5 h, six—and only six—mouse immunoglobulin spots lights up, proving the co-localization of the SWNT labels and the GαM conjugated proteins, and the success of the scheme, as shown in FIG. 3D.

Additionally, selective detection of biotinylated species with SWNT-labeled streptavidin (another frequently used reporting protein), as well as detection performed with 3% FCS, representing a complex biological background, are also observed (data not shown).

### Example 5

### Highly Sensitive Detection by the SWNT-Labeled Protein

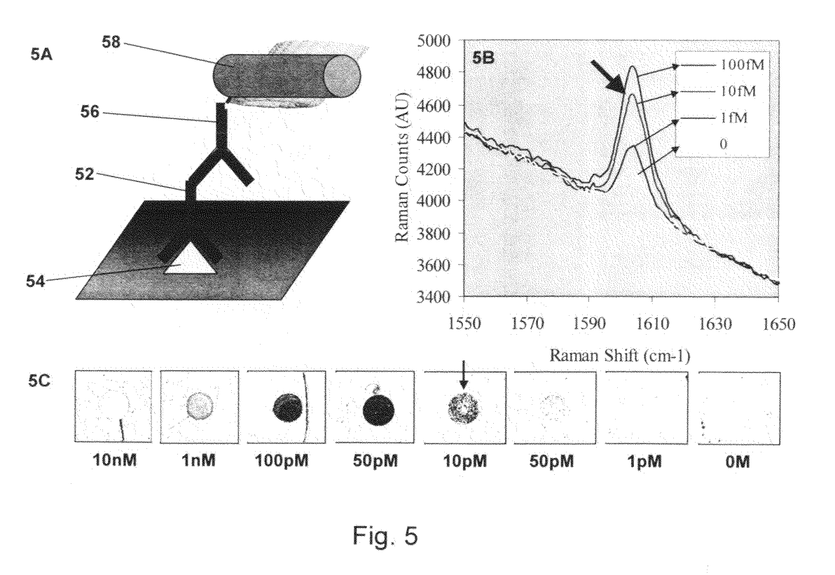

The detection limit of the SWNT-label is determined with an indirect detection format (FIG. 5A). FIG. 5A shows the above-described protein spot with a specific antibody recognizing it, bound to an anti-IgG antibody labeled with a nanotube. Human serum albumin spotted on the surface was incubated with increasing concentrations of MαHSA overnight at 4° C. After rinsing loosely bound materials away 3× with PBS for 30 min, 50 nM of the SWNT-labeled GαM is incubated for 1.5 h. After 3×PBS rinses for 30 min and 1× water rinse for 1 min, the slides were blown dry and the Raman intensities read (FIG. 5B). FIG. 5B shows a clear signal at 10 femtomole. To compare the sensitivity of the Raman detection method with the standard fluorescent technique, the same concentration of cy3-conjugated GαM was incubated instead (FIG. 5C). The results show the SWNT labels, with the detection limit of ˜10 picomole (arrow in 5C), outperformed the fluorescent tags by ˜3 orders of magnitude. Others report fluorescent detection limits to be in the 5-10 pM range as well.

The reasons for the high sensitivity observed are attributed mainly to: (1) The highly sensitive nature of the SWNT Raman signal, as individual SWNTs can be easily detected, enabling fM detection level to be reached and eliminating the need for amplification steps that can further complicated the final results (as required in calorimetric methods). (2) The selectivity of the Raman signal since biological molecules do not show competing Raman backgrounds, thus permitting the detection limit to be lowered. (3) The engineered PEG layer surrounding the SWNTs, which imparts biocompatibility as well as reduces nonspecific binding, as such it allows accurate reports by the SWNT labels to be obtained.

Since the length of the SWNT label, even after cutting, is ˜150 nm, much larger than the dimensions of the proteins (GαM); there is a potential that the modification of proteins with SWNTs will disrupt their binding sites. Though long in length, the nanotube is very small in diameter. In addition, since selective as well as sensitive recognition by the labeled GαM is still obtained as shown in FIG. 5B and FIG. 5C, the biological activity of the GαM must still be relatively well preserved-proving that the presence of the SWNT label does not interfere with the biological functionality of the labeled protein.

### Example 6

### Robustness and Compatibility of the SWNT-Labels

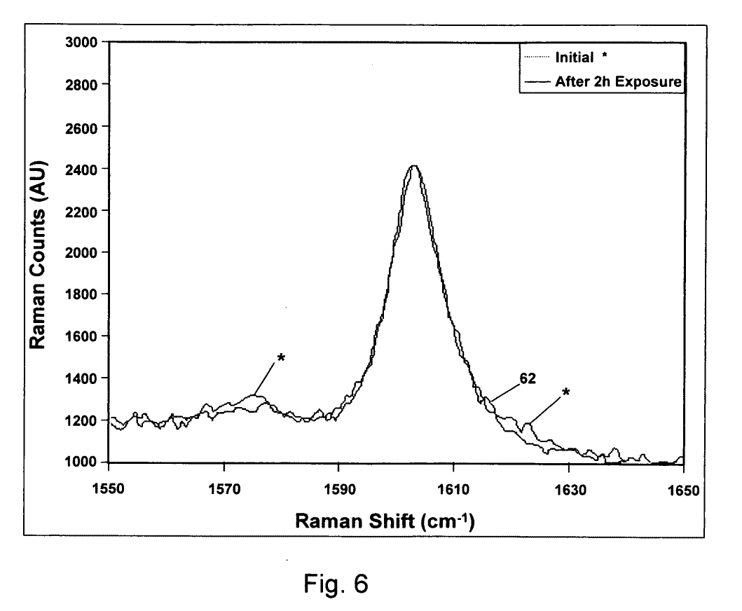

The signal from the SWNT labels is stable during the scanning, as shown in FIG. 6. Even after 2 h of continuous exposure to the Raman laser (a typical scan only lasts 30 s), in ambient light (curve 62), there is no degradation of the signal. This is a distinct advance over fluorescent labels since significant quenching upon prolong exposure of the imaging laser is often observed, and thus allows measurements to be carried out with higher accuracy and reproducibility, with added convenience in sample handling (no need avoid light exposure). This benefit, together with the capability to perform sensing in complex biological background without the need of amplification steps, illustrates the robustness of this novel technique.

The SWNT labels are also compatible with protocols for microarray as demonstrated by FIG. 7. A robotic microarray was used to generate ˜50 nm proteins spots (instead of the hand-made ones in previous figures) on a ProteinReady slide and successful detection of MαHSA by SWNT-labeled GαM secondary antibodies, is shown. The small dimensions of the Raman laser allows high-resolution map to be generated, showing details within the spot. Additionally, successful detection is also attained with other commonly used microarray slides (epoxy, polylysine, data not shown). As such reporting proteins labeled with SWNTs are compatible in its usage with the microarray technique.

The present protocols can include obtaining different samples and spotting them onto slides or microarrays. The samples may be pre-processed, or analyzed as obtained. In the former category are tissue biopsies and cell lysates. On the other hand, serum samples, environmental water samples and the like may be tested as obtained.

### Example 7

### Preparation of Solubilized Nanoparticles (SWNT) with Hydrophobic Fluorophore (FITC) Conjugated to Hydrophilic Polymer (PEG)

This example describes the preparation of a conjugate as illustrated in FIG. 8A. As shown there, FITC will physically adsorb onto the graphene surface of an SWNT, with a covalently linked PEG molecule extending into the medium for attachment to a labeling molecule.

A representative protocol is as follows: 1 mM of Fluor-PEG(5000)-NHS (Nektar Therapeutics) was sonicated with 0.25 mg/mL single-walled carbon nanotubes (SWNTs, as produced HiPco purchased from Carbon Nanotechnologies) for ˜1 hr (protected from light where possible) and centrifuged at ˜25,000 g for 6 hrs. The pellet formed at the bottom of the centrifuge tube containing aggregates, bundles, and impurities was discarded, and the supernatant was collected and filtered through a centrifugal filter device (Millipore Amicon 100k Da molecular cutoff) to remove excess material. Over time, the NHS ester group on Fluor-PEG(5400)—NHS is hydrolyzed and became carboxyl groups. The carboxyl groups can be used for subsequent conjugation to biological molecules.

### Example 8

### Near-Infrared Cellular Imaging of SWNTs Functionalized with PEG and Conjugated to Antibodies Recognizing Cell Surface Markers

Non-specific binding-free, highly water-soluble and biologically inert SWNTs were obtained by functionalization with phospholipid-polyethyleneglycol-amine (PL-PEG-NH₂) with long (˜5,400D) PEG chains, and conjugated with Rituxan® Rituximab, Genentech, Inc., an antibody specific to the CD20 cell surface receptor (hereinafter “Anti-CD 20’). This Anti-CD 20 IgG1 human-murine chimeric monoclonal antibody is indicated for the treatment of non-Hodgkin's lymphoma (NHL), which is a cancer that develops in the lymphatic system. Also used in this example was Herceptin® trastuzumab (hereinafter Anti-erbB2), Genentech, Inc., a humanized recombinant IgG1 monoclonal antibody which recognizes the extracellular segment of the HER2/neu receptor (also known as the erbB2 receptor), a receptor for epidermal growth factor (EGF). This receptor is expressed on various cell types, including tumor epithelial cells. In various tumors, members of the EGF receptor family are overexpressed, leading to abnormal hetero- and homodimer formation and, consequently, to altered ligand binding and aberrant signaling. Her2 is overexpressed in 20-30% of all breast cancer patients, and its overexpression has been correlated with poor prognosis.

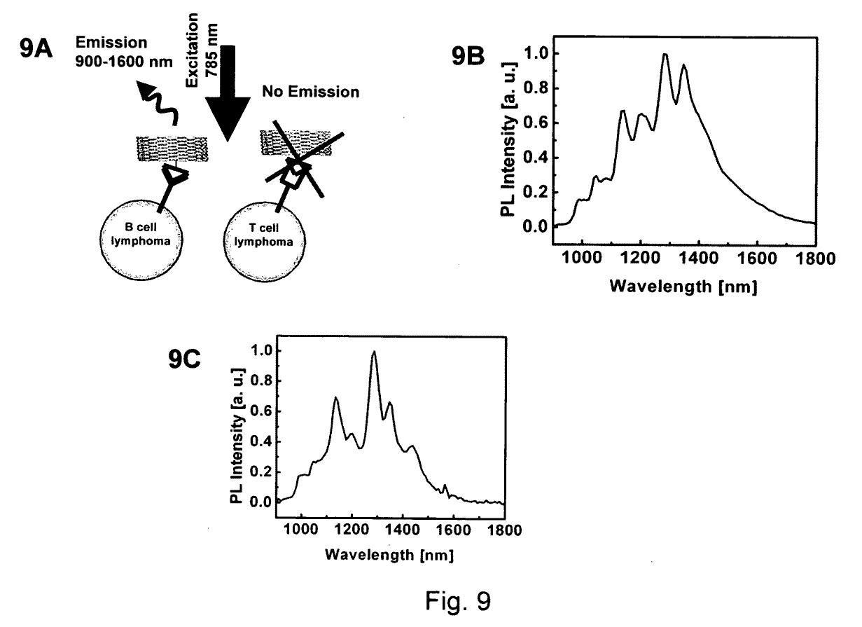

It is shown that SWNT-antibody conjugate selectively bind to cell surface receptors by detecting the intrinsic NIR photoluminescence of SWNTs with very low background autofluorescence. This establishes SWNTs as novel NIR fluorophores for highly sensitive and selective biological detections. Negligible background signals due to autofluorescence of cells in the NIR region were observed compared to SWNT photoluminescence. Little variation in the NIR autofluorescence levels was seen between different cell lines. Highly water-soluble, short (average length ˜50-150 nm) Hipco SWNTs functionalized with phospholipid-polyethyleneglycol-amine (PL-PEG-NH₂) were prepared with long (˜5,400D) PEG chains, and (see below) were found to exhibit very little nonspecific binding. The PEGylated SWNTs were covalently linked via sulfo-SMCC to thiolated Anti-CD 20 or Anti-erbB2. The NIR photoluminescence emission spectrum of the SWNT-Anti-CD 20 conjugate under 785 nm laser excitation showed peak structures in the 1000-1600 nm region (FIG. 9B) due to several SWNTs in the Hipco material with specific chiralities in resonance with the laser.

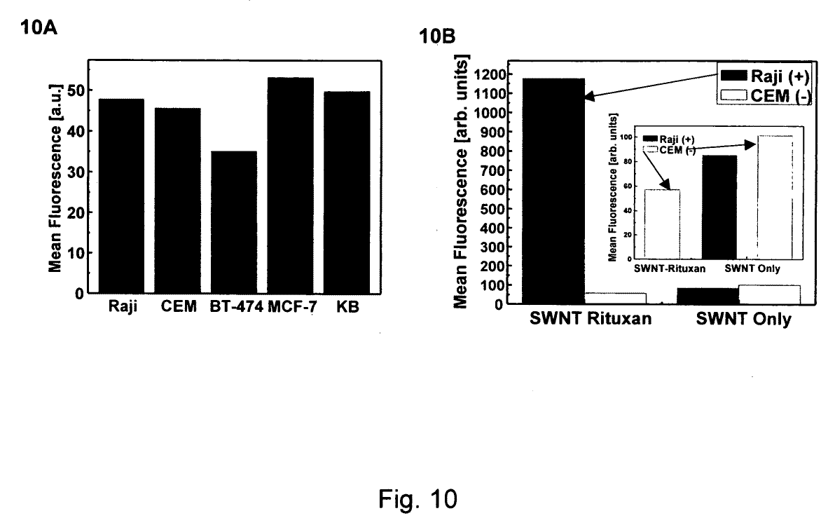

To test the receptor specificity of the anti-CD 20 conjugate, B cells and T cells were incubated in solutions of SWNT-Anti-CD 20 conjugates for 1 h at 4° C. to allow the conjugates to interact with the cell surface but block internalization via endocytosis. The cells were then washed and imaged by detecting NIR photoluminescence from 900 to 2200 nm using an InGaAs detector under 785 nm excitation. We observed bright NIR emission of SWNTs on Raji cells (B cell lymphoma) under NIR fluorescence microscopy, suggesting SWNT-Anti-CD 20 binding to CD20 cell surface receptors on Raji cells. An emission spectrum (FIG. 9C) taken on a Raji cell under high magnification confirmed that the light collected was coming from SWNTs. In contrast, NIR fluorescence images taken on SWNT-Anti-CD 20 incubated CEM cells (T cells, CD20 negative) showed little NIR photoluminescence (FIG. 9A), indicating the lack of SWNT-Anti-CD 20 binding to T cells. Control experiments found that our PEGylated SWNTs without Anti-CD 20 conjugation exhibited low binding to both B cells and T cells (FIG. 10). These results suggest highly specific binding of our SWNT-Anti-CD 20 conjugates to CD20 expressing Raji cells (B cells), revealed by detecting the intrinsic bandgap photoluminescence of SWNTs in the NIR.

To quantify the degree of binding specificity of SWNT-Anti-CD 20 to Raji B cells over CEM T cells, we allowed the cells to assemble densely until near a monolayer for NIR photoluminescence imaging. Under this condition, the mean pixel value of NIR photoluminescence was directly proportional to the average fluorescence of the cells. We found that the positive/negative ratio between the mean photoluminescence levels of SWNT-Anti-CD 20 on Raji and CEM cells was ˜55:1, demonstrating highly selective recognition of CD20 cell surface receptors by SWNT-Anti-CD 20 conjugates and minimal non-specific binding of the conjugates to the negative cells.

The variation of fluorescence intensity over the cells was measured. This was aimed at obtaining information about cell surface receptor variations, and the data resembled what one would obtain using conventional visible fluorescent label in fluorescent-activated cell sorting, or FACS. The Raji sample shows a distribution shifted to higher fluorescence intensity compared to the CEM sample, again indicating higher binding by the positive sample. The mean NIR photoluminescence intensity was at least 700 times greater for the Raji (positive) cells than the CEM (negative control) cells (data not shown). This result illustrated the potential of using SWNTs as NIR labels for cell sorting, much like fluorescent molecules used for FACS in the visible spectrum. Large numbers of cells can be investigated with SWNTs as NIR tags for cell imaging over large cell populations on surfaces, or using regular FACS instruments equipped with a NIR detector.

The SWNT-Anti-erbB2 conjugate was prepared in an analogous manner to the SWNT-Anti-CD 20 conjugate. The positive cell used was the BT-474 cell line, which is HER2/neu positive. The MCF-7 cell line was used as a negative. The results of NIR fluorescence imaging for the positive and negative cell lines were obtained at low cell density for ease of viewing. Mean fluorescence values from a higher density area showed a positive/negative ratio of ˜20:1. The mean fluorescence values for the BT-474 cells were at least 200 times greater than for the MCF-7 (negative control) samples.

A major advantage of using SWNT as an NIR fluorophore was that SWNT photoluminescence occurs in the 1-2 μm region of little or no cellular autofluorescence. In control experiments, we observed negligible background signals due to autofluorescence of cells in the NIR region compared to SWNT photoluminescence. Also important was that little variation in the NIR autofluorescence levels was seen between different cell lines. This was advantageous over high autofluorescence levels with large variations between cells lines in the visible region. As a result, SWNT NIR tags may allow for highly sensitive detection of low expression levels of cell surface proteins, which could be valuable to various biological and medical applications such as disease diagnosis and assessment of response to therapy at the cellular level.

### Methods

### PEGylation of SWNTs by PL-PEG₅₄₀₀-NH₂

Phospholipid (PL)-PEG₅₄₀₀-NH₂ (PEG MW=approx. 5400) at a concentration of 170 μM was sonicated with 0.25 mg/mL of as-produced HiPco SWNTs (Carbon Nanotechnologies, batch R0559) in water for 1 h at room temperature, and the resulting suspension was ultracentrifuged at 200,000 g for 1 h. The aggregate obtained after centrifugation, containing nanotube bundles, catalyst from the HiPco production process, and other impurities, was discarded. Excess PL-PEG₅₄₀₀-NH₂ was removed by filtration through centrifugal 100 kDa filters (Millipore Amicon Ultra) immediately before conjugation to Anti-CD 20. PL-PEG₅₄₀₀-NH₂ was synthesized by reacting NHS-PEG₃₄₀₀-BOC (Nektar) to PL-PEG₂₀₀₀-NH₂, i.e., DSPE-PEG₂₀₀₀-amine (1,2-distearoyl-sn-glycero-3-phosphoethanolamine-N-[amino(polyethylene glycol)₂₀₀₀]) (Avanti Polar Lipids) in methanol, then deprotecting with trifluoroacetic acid.

### Anti-CD 20 Conjugation on SWNTs

Thiolated Anti-CD 20 was conjugated to the amine groups on PEGylated SWNTs via a sulfo-SMCC linker. Rituxan was graciously provided by Drs. Alice Fan and Dean Felsher of the Stanford School of Medicine. The thiolation was done by mixing Anti-CD 20 (10 mg/mL) with Traut's Reagent (Pierce) at a 1:10 molar ratio in phosphate buffered saline (PBS) in the presence of 20 mM EDTA (final pH ˜8) for 2 h. Unreacted Traut's Reagent was removed by filtration through a centrifugal 100 kDa filter (Microcon Ultracel YM-100). The thiolated Anti-CD 20 was used immediately in the following conjugation. MCF-7 and BT-474 cell lines were obtained from the American Type Culture Collection (ATCC). MCF-7 cells were cultured in Dulbecco's Modified Eagle's Medium (DMEM) supplemented with 10% FBS, 100 U/ml penicillin, 100 μg/ml streptomycin and 1% Non-essential amino acid solution while BT-474 cells were grown in DMEM containing 10% FBS, 5 g/l glucose, 100 U/ml penicillin and 100 μg/ml streptomycin.

SWNTs at a concentration of 400 nM functionalized by PL-PEG₅₄₀₀-NH₂ were reacted with 2 mM Sulfo-SMCC (Pierce) for 2 h in PBS at pH 7.4. After thorough removal of excess sulfo-SMCC by filtration through a 100 kDa filter, the SWNT solution was mixed with thiolated Anti-CD 20 in PBS in the presence of 20 mM EDTA. In the reaction buffer, the final SWNT concentration was 300 nM, while that of Anti-CD 20 was 3 μM (˜1:10 molar ratio). The reaction was allowed to proceed overnight at 4° C., affording the SWNT-Anti-CD 20 conjugate. This conjugate was used directly without further treatment. A similar procedure may be used for other antibodies.

### AFM Characterization of Phospholipid-Functionalized SWNTs

A clean silicon oxide substrate was soaked in 2.5 mM (3-aminopropyl) triethoxysilane (Aldrich) for 5 min, cleaned with deionized water, blow dried under air, then soaked in PL-PEG₅₄₀₀-NH₂ functionalized SWNTs at a concentration of 40 nM for 30 min. Images were acquired using a tapping mode AFM (Digital Instruments NanoScope IIIa) at room temperature under ambient conditions (FIG. 1C). The lengths of 100 SWNTs were measured, giving an average SWNT length of ˜83 nm. The average SWNT diameter was found to be 1.6 nm, which is consistent with HiPco SWNTs (average diameter 0.7-1.1 nm) that have been well PEGylated.

### Cell Culture

CEM.NK T-cell line was obtained from the NIH Aids Reagents Program. Raji B-cell line was a kindly provided by Drs. Alice Fan and Dean Felsher. These cells were grown in RPMI-1640 supplemented with 10% fetal bovine serum (FBS), 100 U/ml penicillin and 100 μg/ml streptomycin. All the cell culture related reagents were purchased from Invitrogen.

The MCF-7 cell line is available as ATCC HTB 22. The BT-474 cell line may be obtained from Lawrence Berkeley National Laboratory. Raji cells may be obtained as ATCC CCL 86. KB cells may be obtained as ATCC CCL17.

For the cell incubation, 400 μl of cells (1 million/ml) were incubated with 100 μl of PL-PEG₅₄₀₀-NH₂ functionalized SWNTs with or without Anti-CD 20 conjugation in PBS for 1 hour at 4° C. The final SWNT concentration in the solution during incubation was ˜20 nM. Cells were washed with PBS 3 times to remove unbound nanotubes before used for NIR photoluminescence imaging.

### NIR Photoluminescence Imaging of Cells

For NIR photoluminescence imaging, cell samples prepared as described above were placed in Nunc Lab-Tek Chambered Coverglass with a thin 1.0 borosilicate glass optical window. For high resolution images, imaging scans were performed ˜15 minutes after addition of cells to the sample holder, yielding a sub-monolayer. The sub-monolayer was imaged for ease of viewing. For low resolution imaging and statistical evaluation, the cells were allowed to sit in the sample holder for at least 1 hour, so that at least one monolayer had formed. When the sample was prepared in this way, the entire scan area was covered with cells.

All NIR fluorescence images were taken using a home built inverted NIR fluorescence microscope. Excitation from 10 mW diode laser at 785 nm (Renishaw) was focused using a 100× IR coated objected lens (Olympus). The laser spot size width on the sample was about 4 microns FWHM. Emitted light was collected through the same objective and collected using an OMA-V 1024 element linear InGaAs array (Princeton Instruments), Integration time was 250 milliseconds and background subtraction was performed to eliminate dark counts. Spectra were taken using a Princeton Instruments SP2300i spectrometer.

Images were taken by scanning the sample with a 3D motorized translation stage (Newport). For low resolution images, which were used to gather statistics, the sample was scanned over a 200 by 200 micron area with 5 micron steps in both the horizontal and vertical directions. The signal from each point was binned to give the value of a pixel. For high resolution images, line scans were taken to reduce measurement time without sacrificing resolution. Each line was 4 microns wide (to correspond with the laser spot) and 1 micron high, yielding 0.5 by 1 micron pixels. The image was corrected post collection to account for the intensity profile of the laser spot.

### Treatment of Background

Since the sample holder used had a glass optical window, there was a small background fluorescence. The approximate level of the background fluorescence was found by taking a NIR fluorescence image of the sample holder without any cell material. This yielded a mean NIR fluorescence value of 90±5 counts, and was found to be repeatable for all sample holders. This value was subtracted from the data to give relevant statistics about the effectiveness SWNT binding. It was also observed that the cells had a small autofluorescence signal, which variedly only slightly for four different cell lines tested: Raji; CEM, BT-474, MCF-7 human breast adenocarcinoma cell line, and KB human epidermoid carcinoma cell line. The background values were also subtracted from the shown data.

### NSB Test

Control experiments were performed to confirm that the positive signal seen was a result of detection of the cell surface receptor. The SWNT-Anti-CD 20 treated positive (Raji) cells show high NIR fluorescence signal. When Raji cells were treated with PEGylated SWNTs without the Anti-CD 20 antibody, the signal was comparable to that seen for the negative (CEM) cell line. This confirms that the signal seen from the positive line is from recognition of the cell surface receptor, rather than preferential non-specific binding.

### CONCLUSION

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

### REFERENCES

1. He, P. A.; Xu, Y.; Fang, Y. Z., “Applications of carbon nanotubes in electrochemical DNA biosensors,” Microchimica Acta 2006, 152, (3-4), 175.
2. Chen, R. J.; Choi, H. C.; Bangsaruntip, S.; Yenilmez, E.; Tang, X. W.; Wang, Q.; Chang, Y. L.; Dai, H. J., “An Investigation of the Mechanisms of Electronic Sensing of Protein Adsorption on Carbon Nanotube Devices,” Journal of the American Chemical Society 2004, 126, (5), 1563.
3. Chen, R J.; Bangsaruntip, S.; Drouvalakis, K. A.; Kam, N. W. S.; Sbim, M.; Li, Y. M.; Kim, W.; Utz, P. I; Dai, H. J., “Noncovalent functionalization of carbon nanotubes for highly specific electronic biosensors,” Proceedings of the National Academy of Sciences of the United States of America 2003, 100, (9), 4984.
4. Kam, N. W. S.; Liu, Z. A.; Dai, H. J., “Carbon nanotubes as intracellular transporters for proteins and DNA: An investigation of the uptake mechanism and pathway,” Angewandte Chemie—International Edition 2006, 45, (4), 577.
5. Kam, N. W. S.; Jessop, T. C.; Wender, P. A.; Dai, H. J., “Nanotube molecular transporters: Internalization of carbon nanotube-protein conjugates into mammalian cells,” Journal of the American Chemical Society 2004, 126, (22), 6850.
6. Kam, N. W. S.; Dai, H. J., “Carbon nanotubes as intracellular protein transporters: Generality and biological functionality,” Journal of the American Chemical Society 2005, 127, (16), 6021.
7. Mattson, M. P.; Haddon, R. C.; Rao, A. M., “Molecular functionalization of carbon nanotubes and use as substrates for neuronal growth,” Journal of Molecular Neuroscience 2000, 14, (3), 175.
8. Lovat, V.; Pantarotto, D.; Lagostena, L.; Cacciari, B.; Grandolfo, M.; Righi, M.; Spalluto, G.; Prato, M.; Ballerini, L., “Carbon nanotube substrates boost neuronal electrical signaling,” Nano Letters 2005, 5, (6), 1107.
9. Hui, H.; Yingchun, N.; Montana, V.; Haddon, R. C.; Parpura, V., “Chemically functionalized carbon nanotubes as substrates for neuronal growth,” Nano Letters 2004, 4, (3), 507.
10. Zheng, M.; Jagota, A.; Semke, E. D.; Diner, B. A.; McLean, R S.; Lustig, S. R; Richardson, R. E.; Tassi, N. G., “DNA-assisted dispersion and separation of carbon nanotubes,” Nature Materials 2003, 2, (5), 338.
11. O'Connell, M. J.; Bachilo, S. M.; Huffman, C. B.; Moore, V. c.; Strano, M. S.; Haroz, E. H.; Rialon, K. L.; Boul, P. J.; Noon, W. H.; Kittrell, c.; Ma, J. P.; Hauge, R H.; Weisman, R. B.; Smalley, R E., “Band gap fluorescence from individual single-walled carbon nanotubes,” Science 2002, 297, (5581), 593.
12. Kam, N. W. S.; Liu, Z.; Dai, H. J., “Functionalization of carbon nanotubes via cleavable disulfide bonds for efficient intracellular delivery of siRNA and potent gene silencing,” Journal of the American Chemical Society 2005, 127, (36), 12492.
13. Dwyer, C.; Guthold, M.; Falvo, M.; Washburn, S.; Superfine, R.; Erie, D., “DNA-functionalized singlewalled carbon nanotubes,” Nanotechnology 2002, 13, (5), 601.
14. Chen, R J.; Zhan, Y. G.; Wang, D. W.; Dai, H. J., “Noncovalent sidewall functionalization of singlewalled carbon nanotubes for protein immobilization,” Journal of the American Chemical Society 2001, 123, (16), 3838.
15. Jorio, A.; Saito, R; Hafner, J. H.; Lieber, C. M.; Hunter, M.; McClure, T.; Dresselhaus, G.; Dresselhaus, M. S., “Structural (n,m) determination of isolated single-wall carbon nanotubes by resonant Raman scattering,” Physical Review Letters 2001, 86, (6), 1118.

What is claimed is:

1. A composition comprising:
a) a carbon nanoparticle;
b) a solubilizing molecule having (i) a hydrophobic portion attached to the carbon nanoparticle and (ii) a hydrophilic portion for solubilizing the composition and having a linking group; and
c) a biological labeling molecule attached to the linking group.

2. The composition of claim 1 where the carbon nanoparticle is a carbon nanotube having predominantly sp2 bonded carbon.

3. The composition of claim 1 where the carbon nanoparticle is an SWNT.

4. The composition of claim 1 where the carbon nanoparticle is an MWNT.

5. The composition of claim 1 where the amphiphilic molecule comprises a phospholipid.

6. The composition of claim 1 where the amphiphilic molecule comprises PEG.

7. The composition of claim 1 where the amphiphilic molecule comprises a hydrophobic portion selected from the group consisting of: an aromatic compound, an aliphatic compound, and a polycyclic aromatic compound.

8. The composition of claim 7 where the polycyclic aromatic compound is a pyrene.

9. The composition of claim 1 where the amphiphilic molecule comprises a fluorophore adsorbed on the nanoparticle.

10. The composition of claim 9 where the fluorophore is a fluorescein derivative selected from the group consisting of FITC, DTAF and NHS-fluorescein.

11. The composition of claim 10 where the amphiphilic molecule comprises FITC-PEG-NH2.

12. The composition of claim 1 where the biological labeling molecule is selected from the group consisting of proteins and polynucleotides.

13. The composition of claim 11 where the biological labeling molecule is an antibody.

14. A method for preparing a composition for labeling a biological material, comprising the steps of:
(a) obtaining a nanoparticle;
(b) attaching the nanoparticle to a solubilizing molecule having a hydrophilic portion; and
(c) linking a biological labeling molecule to the hydrophilic portion.

15. The method of claim 14 where the nanoparticle is selected from the group consisting of fullerenes, SWNTs, DWNTs and MWNTs.

16. The method of claim 14 where the solubilizing molecule comprises PEG.

17. The method of claim 14 where the PEG is covalently linked to the nanoparticle.

18. The method of claim 14 further comprising the step of adding additional PEG units to the PEG portion.

19. The method of claim 15 where the solubilizing molecule comprises a phospholipid linked to PEG.

20. The method of claim 15 where the biological labeling molecule is an antibody or a polynucleotide.

21. The method of claim 15 where the biological labeling molecule is a thiolated antibody, and comprising the step of linking the thiolated antibody to a bifunctional coupling agent which has been bound to the solubilizing molecule.

22. The method of claim 15 where the nanoparticles is a carbon nanotube.

23. The method of claim 22 where the carbon nanotube is selected from the group consisting of SWNTs, DWNTs and MWNTs.

24. A method of detecting an analyte using Raman scattering with an optical source exciting a sample and an optical detector detecting changes in light scattered by the sample, comprising the step of labeling a portion of the sample with a specific ligand linked to a carbon nanoparticle.

25. The method of claim 24 where the detecting detects a Raman shift around 1550-1600 cm-1.

26. The method of claim 24 where the detecting detects a Raman shift around 0-400 cm-1.

27. The method of claim 24 where the nanoparticle is linked to an amphiphilic molecule.

28. The method of claim 24 where the carbon nanoparticles is an SWNT.

29. The method of claim 24 where the analyte is present in a concentration less than 10 fM.

30. The method of claim 24 where the analyte and ligand is selected from the group consisting of antigen-antibody and nucleic acids and polynucleotides.

31. The method of claim 24 where the step of labeling a portion of the sample with a specific ligand linked to a carbon nanoparticle comprises the step of labeling the sample with a specific ligand linked to a fluorophore and a carbon nanoparticles, and further comprising the step of detecting fluorescence.

32. A method of labeling a cell for detection by fluorescence, comprising the steps of:
a) contacting the cell with a composition comprising
b) a carbon nanoparticle;
c) a solubilizing molecule having a hydrophobic portion attached to the carbon nanoparticle and a hydrophilic portion for solubilizing the composition and having a linking group; and
d) a biological labeling molecule specific for the cell attached to the linking group;
e) forming a complex of the cell and the composition of step (a);
f) irradiating the complex with near infrared light; and
g) detecting near infrared photoluminescence from the carbon nanoparticle.

33. The method of claim 32 where the carbon nanoparticle is a carbon nanotube and the photoluminescence is in the 900-1600 nm region.

34. The method of claim 32 where the irradiating is done with a near infrared laser.

35. The method of claim 34 further comprising the step of measuring Raman scattering.

36. The method of claim 32 where the biological labeling molecule is a monoclonal antibody to a cell surface antigen.

37. A composition comprising:
a) at least two different carbon nanoparticles;
b) solubilizing molecules having (i) a hydrophobic portion attached to the carbon nanoparticles and (ii) a hydrophilic portion for solubilizing the composition and having a linking group; and
c) at least two different biological labeling molecule attached to the linking group of respective different nanoparticles.

38. A method of detecting analytes in a sample comprising the steps of applying the composition of claim 32 to the analytes, contacting the sample with coherent light, and identifying Raman signals from the sample, where said two different biological labeling molecules produce two different Raman signals upon detection of two different analytes.
